(19) United States
(12) Patent Application Publication
(10) Pub. No.: US 2024/0090480 A1
(43) Pub. Date: Mar. 21, 2024

(54) SPLIT-INTEIN METHODS AND COMPOSITIONS FOR GENERATION OF SINGLE SEX OFFSPRING

(71) Applicant: Joseph Fenton LAWLER, Spicewood, TX (US)

(72) Inventor: Joseph Fenton LAWLER, Spicewood, TX (US)

(21) Appl. No.: 18/467,024

(22) Filed: Sep. 14, 2023

Related U.S. Application Data
(60) Provisional application No. 63/376,063, filed on Sep. 16, 2022.

(51) Int. Cl. A01K 67/027

(52) U.S. Cl. CPC A01K 67/0275; A01K 2217/30; A01K 2227/30

(57) ABSTRACT

Described herein are methods and compositions for generating single sex offspring using split-intein approach. In particular, methods and compositions are provided to generate single sex and genetically modified offspring. These techniques can be applied to compassionate animal breeding.

### CROSS REFERENCE

This application claims the benefit of U.S. Provisional Application No. 63/376,063, filed Sep. 16, 2022, which is incorporated herein by reference in its entirety.

### BACKGROUND

In many agricultural applications it is desirable to generate single sex offspring. For example, the products of a mating between two chicken lines optimized for egg laying characteristics are only useful when offspring are female because males cannot lay eggs and breeds used for egg laying are generally not optimized for meat production. As a result, male chicks are separated from females as soon after hatching as possible and culled. Because male and female chicks do not look appreciably different for several days after hatching, specialized methods must be employed to distinguish them. One of the oldest methods involves the use of chicken sexers. These are skilled individuals who are able to determine the sex of a chick before it is obvious to an untrained person. Still, despite their acumen, chicken sexers are not perfect. Moreover, even if chicken sexers were perfectly accurate, they still must wait for the egg to hatch which means eggs harboring males must be carried through the entire incubation process before being identified and culled. This wastes resources on eggs that are not suitable for producing laying hens. Thus, finding new method to generate single sex offspring of animal such as chicken is needed.

### SUMMARY OF THE INVENTION

In aspects, provided herein are non-human vertebrate animals comprising one or more nucleotide sequences. In some embodiments, the one or more nucleotide sequences comprise a promoter, a split intein fragment, and a toxin fragment. In some embodiments, the one or more nucleotide sequences are integrated in one or more chromosomes. In some embodiments, the one or more chromosomes is an allosome. In some embodiments, the one or more chromosomes is an autosome. In some embodiments, the split intein fragment is fused to the toxin fragment. In some embodiments, the toxin fragment is a toxin selected from the group consisting of a nuclease, a ribosome toxin, and a protease. In some embodiments, the nuclease comprises Barnase, an RNase, or a restriction endonuclease. In some embodiments, the ribosome toxin comprises diphtheria, ricin, abrin, or pokeweed antiviral protein. In some embodiments, the protease comprises a caspase, proteinase K, trypsin, chymotrypsin, or papain. In some embodiments, the non-human vertebrate animal is selected from the group consisting of cow, mouse, rat, rabbit, guinea pig, chicken, fish, bird, reptile, camelid, bovine, chimpanzee, sheep, goat, and non-human primate.

In additional aspects, there are provided non-human vertebrate animals comprising one or more nucleotide sequences, wherein the one or more nucleotide sequences comprises a promoter, a first split intein fragment, a first toxin fragment, or any combination thereof. In some embodiments, the one or more nucleotide sequences further comprises an intron. In some embodiments, the one or more nucleotide sequences are integrated in one or more chromosomes in the non-human vertebrate animal. In some embodiments, the chromosome is an autosome. In some embodiments, the chromosome is an allosome. In some embodiments, the first split intein fragment is fused to the first toxin fragment. In some embodiments, the first split intein fragment comprises an N-terminal intein. In some embodiments, the first split intein fragment comprises a C-terminal intein. In some embodiments, the first toxin fragment comprises an N-terminal toxin. In some embodiments, the first toxin fragment comprises a C-terminal toxin. In some embodiments, the first toxin fragment is a toxin selected from the group consisting of a nuclease, a ribosome toxin, and a protease. In some embodiments, the nuclease comprises Barnase, an RNase, or a restriction endonuclease. In some embodiments, the ribosome toxin comprises diphtheria, ricin, abrin, or pokeweed antiviral protein. In some embodiments, the protease comprises a caspase, proteinase K, trypsin, chymotrypsin, or papain. In some embodiments, the promoter is normally inactive in an adult of the non-human vertebrate animal. In some embodiments, the promoter is active during embryogenesis.

In further aspects, there are provided non-human vertebrate animals comprising one or more nucleotide sequences, wherein the one or more nucleotide sequences comprises a promoter, a second split intein fragment, a second toxin fragment, or any combination thereof. In some embodiments, the one or more nucleotide sequences further comprise an intron. In some embodiments, the one or more nucleotide sequences are integrated in one or more chromosomes in the non-human vertebrate animal. In some embodiments, the chromosome is an autosome. In some embodiments, the chromosome is an allosome. In some embodiments, the second split intein fragment is fused to the second toxin fragment. In some embodiments, the second split intein fragment comprises an N-terminal intein. In some embodiments, the second split intein fragment comprises a C-terminal intein. In some embodiments, the second toxin fragment comprises an N-terminal toxin. In some embodiments, the second toxin fragment comprises a C-terminal toxin. In some embodiments, the second toxin fragment is a toxin is selected from the group consisting of a nuclease, a ribosome toxin, and a protease. In some embodiments, the nuclease comprises Barnase, an RNase, or a restriction endonuclease. In some embodiments, the ribosome toxin comprises diphtheria, ricin, abrin, or pokeweed antiviral protein. In some embodiments, the protease comprises a caspase, proteinase K, trypsin, chymotrypsin, or papain.

In another aspect, there are provided pluralities of non-human vertebrate animals each comprising one or more nucleotide sequences integrated in one or more chromosomes, wherein a first non-human vertebrate animal comprises a first split intein fragment and a first toxin fragment, and a second non-human vertebrate animal comprises a second split intein fragment, and a second toxin fragment. In some embodiments, the chromosome is an autosome. In some embodiments, the chromosome is an allosome. In some embodiments, the first split intein fragment comprises an N-terminal intein. In some embodiments, the first split intein fragment comprises a C-terminal intein. In some embodiments, the first toxin fragment comprises an N-terminal toxin. In some embodiments, the first toxin fragment comprises a C-terminal toxin. In some embodiments, the second split intein fragment comprises an N-terminal intein. In some embodiments, the second split intein fragment comprises a C-terminal intein. In some embodiments, the second toxin fragment comprises an N-terminal toxin. In some embodiments, the second toxin fragment comprises a C-terminal toxin. In some embodiments, the first toxin fragment and/or the second toxin fragment is a toxin is selected from the group consisting of a nuclease, a ribosome toxin, and a protease. In some embodiments, the nuclease comprises Barnase, an RNase, or a restriction endonuclease. In some embodiments, the ribosome toxin comprises diphtheria, ricin, abrin, or pokeweed antiviral protein. In some embodiments, the protease comprises a caspase, proteinase K, trypsin, chymotrypsin, or papain. In some embodiments, the one or more nucleotide sequences in the first non-human vertebrate animal further comprises a promoter. In some embodiments, the promoter is normally inactive in the adult non-human vertebrate animal. In some embodiments, the promoter is active during embryogenesis. In some embodiments, the promoter is activated by a transcription factor.

In another aspect, there are provided non-human vertebrate animals comprising one or more nucleotide sequences, wherein the one or more nucleotide sequences comprises a promoter and a toxin or a fragment thereof, wherein the promoter is activated upon binding of a transcription factor. In some embodiments, the one or more nucleotide sequences further comprise an intron. In some embodiments, the toxin or the fragment thereof is a toxin selected from the group consisting of a nuclease, a ribosome toxin, and a protease. In some embodiments, the nuclease comprises Barnase, an RNase, or a restriction endonuclease. In some embodiments, the ribosome toxin comprises diphtheria, ricin, abrin, or pokeweed antiviral protein. In some embodiments, the protease comprises a caspase, proteinase K, trypsin, chymotrypsin, or papain. In some embodiments, the one or more nucleotide sequences are integrated in one or more chromosomes in the non-human vertebrate animal. In some embodiments, the chromosome is an autosome. In some embodiments, the chromosome is an allosome.

In a further aspect, there are provided non-human vertebrate animals comprising one or more nucleotide sequences, wherein the one or more nucleotide sequences comprises a promoter and a transcription factor. In some embodiments, the one or more nucleotide sequences further comprise an intron. In some embodiments, the one or more nucleotide sequences are integrated in one or more chromosomes in the non-human vertebrate animal. In some embodiments, the chromosome is an autosome. In some embodiments, the chromosome is an allosome.

In another aspect, there are provided non-human vertebrate animals having a genotype comprising a pair of chromosomes, wherein the chromosome comprises a toxin fragment, wherein the toxin fragment is not functional. In some embodiments, the chromosome is an autosome. In some embodiments, the chromosome is an allosome. In some embodiments, the toxin fragment comprises an N-terminal toxin. In some embodiments, the toxin fragment comprises a C-terminal toxin. In some embodiments, the toxin fragment is a toxin selected from the group consisting of a nuclease, a ribosome toxin, and a protease. In some embodiments, the nuclease comprises Barnase, an RNase, or a restriction endonuclease. In some embodiments, the ribosome toxin comprises diphtheria, ricin, abrin, or pokeweed antiviral protein. In some embodiments, the protease comprises a caspase, proteinase K, trypsin, chymotrypsin, or papain.

In additional aspects, there are provided non-human vertebrate animals having a genotype comprising a pair of heterozygous chromosomes, wherein the chromosome comprises a promoter and a toxin or a fragment thereof, wherein the promoter is activated upon binding of a transcription factor. In some embodiments, the chromosome is an autosome. In some embodiments, the chromosome is an allosome. In some embodiments, the toxin or the fragment thereof is a toxin selected from the group consisting of a nuclease, a ribosome toxin, and a protease. In some embodiments, the nuclease comprises Barnase, an RNase, or a restriction endonuclease. In some embodiments, the ribosome toxin comprises diphtheria, ricin, abrin, or pokeweed antiviral protein. In some embodiments, the protease comprises a caspase, proteinase K, trypsin, chymotrypsin, or papain.

In another aspect, there are provided methods of producing a single sex population of non-human vertebrate animals. In some embodiments the method comprises crossing (i) a first non-human vertebrate animal having a first genotype comprising a pair of heterozygous chromosomes comprising one or more nucleotide sequences encoding a first toxin fragment with (ii) a second non-human vertebrate animal having a second genotype comprising a pair of homozygous chromosomes comprising one or more nucleotide sequences encoding a second toxin fragment. In some embodiments, a resulting progeny having a genotype comprising the first toxin fragment and the second toxin fragment is not viable; thereby creating a single sex population. In some embodiments, the chromosome is an autosome. In some embodiments, the chromosome is an allosome. In some embodiments, the non-human vertebrate animal is selected from the group consisting of cow, mouse, rat, rabbit, guinea pig, chicken, fish, bird, reptile, camelid, bovine, chimpanzee, sheep, goat, and non-human primate. In some embodiments, the first toxin fragment and/or the second toxin fragment is a toxin selected from the group consisting of a nuclease, a ribosome toxin, and a protease. In some embodiments, the nuclease comprises Barnase, an RNase, or a restriction endonuclease. In some embodiments, the ribosome toxin comprises diphtheria, ricin, abrin, or pokeweed antiviral protein. In some embodiments, the protease comprises a caspase, proteinase K, trypsin, chymotrypsin, or papain.

In a further aspect, there are provided methods of producing a single sex population of non-human vertebrate animals, comprising the steps of: (a) obtaining (i) a first non-human vertebrate animal, wherein one or more chromosomes comprise a first expression cassette comprising the following elements in 5′ to 3′ orientation: a first promoter, operatively linked thereto a nucleotide sequence encoding for a first toxin fragment and a first split intein fragment; (b) obtaining (ii) a second non-human vertebrate animal, wherein one or more allosomes comprise a second expression cassette comprising the following elements in 5′ to 3′ orientation: a second promoter, operatively linked thereto a nucleotide sequence encoding for a second toxin fragment and a second split intein fragment; (c) crossing the first non-human vertebrate animal and the second non-human vertebrate animals, wherein a resulting progeny containing the first expression cassette and the second expression cassette is not viable; thereby creating a single sex population. In some embodiments, the chromosome is an autosome. In some embodiments, the chromosome is an allosome. In some embodiments, the first split intein fragment comprises an N-terminal intein. In some embodiments, the first split intein fragment comprises a C-terminal intein. In some embodiments, the first toxin fragment comprises an N-terminal toxin. In some embodiments, the first toxin fragment comprises a C-terminal toxin. In some embodiments, the second split intein fragment comprises an N-terminal intein. In some embodiments, the second split intein fragment comprises a C-terminal intein. In some embodiments, the second toxin fragment comprises an N-terminal toxin. In some embodiments, the second toxin fragment comprises a C-terminal toxin. In some embodiments, the first toxin fragment and/or the second toxin fragment is a toxin is selected from the group consisting of a nuclease, a ribosome toxin, and a protease. In some embodiments, the nuclease comprises Barnase, an RNase, or a restriction endonuclease. In some embodiments, the ribosome toxin comprises diphtheria, ricin, abrin, or pokeweed antiviral protein. In some embodiments, the protease comprises a caspase, proteinase K, trypsin, chymotrypsin, or papain. In some embodiments, the first promoter is normally inactive in the non-human vertebrate animal. In some embodiments, the first promoter is active during embryogenesis. In some embodiments, the expression cassette in the first non-human vertebrate animal further comprises a terminator sequence. In some embodiments, the expression cassette in the second non-human vertebrate animal further comprises a terminator sequence.

In another aspect, there are provided processes of producing a non-human vertebrate animal or parts thereof expressing a trait of interest, the trait having a controlled distribution of the trait to progeny, thereby creating a single sex population, wherein the process comprises: (a) obtaining (i) a first non-human vertebrate animal or a cell thereof having in homologous chromosomes, a first nucleotide sequence comprising a first fragment of a nucleotide sequence encoding the trait of interest; (b) obtaining (ii) a second non-human vertebrate animal or a cell thereof having in a second locus of the chromosome heterozygous to the chromosome of step (i), a second nucleotide sequence comprising a second fragment of the nucleotide sequence encoding the trait of interest; and (c) crossing the first non-human vertebrate animals or cells thereof and the second non-human vertebrate animals or cells thereof to generate progeny exhibiting the trait of interest due to binding between a protein or polypeptide encoded by the first nucleotide sequence and a protein or polypeptide encoded by the second nucleotide sequence, wherein the genotype expressing both the first nucleotide sequence and second nucleotide sequence is not viable. In some embodiments, the chromosome is an autosome. In some embodiments, the chromosome is an allosome.

In another aspect, there are provided methods of producing a single sex population of non-human vertebrate animals, comprising the steps of: (a) obtaining (i) a first non-human vertebrate comprising one or more nucleotide sequences integrated in one or more chromosomes comprises the following elements in 5′ to 3′ orientation: a first inactive promoter, operatively linked thereto a nucleotide sequence encoding for a toxin; (b) obtaining (ii) a second non-human vertebrate animal comprising one or more nucleotide sequences integrated in one or more chromosomes comprising the following elements in 5′ to 3′ orientation: a second promoter, operatively linked thereto a transcription factor corresponding to the inactive promoter; and (c) crossing the first non-human vertebrate animal and the second non-human vertebrate animals, wherein a resulting progeny, containing the first inactive promoter, the second promoter expressing the transcription factor corresponding to the first inactive promoter, and the corresponding transcription factor, is not viable; thereby creating a single sex population. In some embodiments, the chromosome is an autosome. In some embodiments, the chromosome is an allosome. In some embodiments, the toxin is selected from the group consisting of a nuclease, a ribosome toxin, and a protease. In some embodiments, the nuclease comprises Barnase, an RNase, or a restriction endonuclease. In some embodiments, the ribosome toxin comprises diphtheria, ricin, abrin, or pokeweed antiviral protein. In some embodiments, the protease comprises a caspase, proteinase K, trypsin, chymotrypsin, or papain.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

An understanding of the features and advantages of the present invention will be obtained by reference to the following detailed description that sets forth illustrative embodiments, in which the principles of the invention are utilized, and the accompanying drawings of which:

FIG. 1 depicts a genetic cross diagram showing how to generate single sex offspring in the third generation, e.g., layer hens, using protein splicing process involving split-intein system.

FIG. 2 depicts an example of DNA construct for generation of single sex offspring. The DNA construct comprises a promoter, a split intein fragment, and a toxin fragment. In some instances, the DNA construct also comprises an intron encoding sequence to facilitate the expression of the split intein fragment and toxin fragment.

FIG. 3 depicts the Punnett Square of possible genotypic outcomes of 3^{rd} generation from genetic crossing of second generation (Z²W and Z¹Z¹) chicken.

FIG. 4 depicts the survival of third generation of chicken. The Z¹W females are viable since Z¹Z² males express both halves of the intein-toxin fusion protein, which kill the cells.

FIG. 5 depicts a schematic of another genetic cross diagram to generate single sex offspring. Instead of using protein splicing process involving split-intein as shown in FIG. 1, this schematic uses a toxin gene driven by a promoter that is not normally active in chicken.

### DETAILED DESCRIPTION OF THE INVENTION

In many agricultural applications, generation of single sex offspring, for example, only female hens, is desirable. The products of a mating between two chicken lines optimized for egg laying characteristics are useful when offspring are female because males cannot lay eggs and are generally not optimized for meat production. As a result, male chicks are separated from females as soon after hatching as possible and culled.

According to the statistical report from United States Department of Agriculture (USDA), U.S. egg production totaled 8.67 billion eggs during June 2022, and the total layer hens in the U.S. on Jul. 1, 2022 is about 366 million (USDA. Chickens and Eggs. July, 2022. ISSN: 1948-9064). Layer hens, however, can lay eggs from 18-19 weeks to 72-78 weeks of age. The layer hen industry, thus, requires the replacement of layer hens annually. Each year, approximately 221.6 million layer hens must be replaced. Assuming an equal sex ratio, this means 523 million layer hen eggs must be hatched of which half will be male. All male chicks will be culled. Each year, up to 300 million male chicks are killed in the U.S., and as many as 7 billion male chicks are culled globally. This presents both animal welfare and ethical issues. In fact, Germany has recently banned the culling of day-old male chicks and Italy plans to follow suit. Companies have also taken notice and recently announced they oppose the practice.

Because male and female chicks do not look appreciably different for several days after hatching, and specialized methods must be employed to distinguish them. One of the oldest methods involves the use of chicken sexers. These are skilled individuals who are able to determine the sex of a chick before it is obvious to an untrained person. Still, despite their acumen, chicken sexers are not perfect, although some can reach greater than 90% accuracy.

Moreover, even if chicken sexers were perfectly accurate, they still must wait for the egg to hatch and grown for several days, which means eggs harboring males must be carried through the entire incubation process before being identified and culled. This wastes resources on eggs which eventually must be culled because male chicks are not suitable for the intended purpose.

Other methods have been developed to separate male and female chicks that rely on feather color (Gohler, D. et al. 2017. Poult Sci. 1; 96(1):1-4). Some methods involve the expression of marker proteins such as green fluorescent protein. Although selectively expressing green fluorescent protein will allow distinguishing between sexes will also result in genetically modified birds that may not be suitable to customers and are subject to greater regulation (Lee, H. et al. 2019. FASEB J. 33(7):8519-8529).

Methods to allow for selection at the egg stage have also been developed. These methods include detecting minute amounts of estrogen, DNA sequences and other analytes that identify the bird's sex (M-E Krautwald-Junghanns, M. et al. 2018. Poult Sci. 1; 97(3):749-757). These methods, while effective often require additional machinery for implementation and do not escape the fact that male eggs must be incubated to some point.

The present disclosure provides methods and compositions whereby eggs that would otherwise bear male chickens fail to develop by utilizing protein splicing mechanism that involves split inteins system or by utilizing promoter driven toxin under specific transcription factor. In some instances, the promoter driven toxin is an inducible promoter. This approach would improve efficiency in a number of ways. Eggs that would otherwise bear male chicks will be suppressed during embryogenesis thereby increasing egg hatching capacity significantly. In addition, no screening method would need to be implemented on the eggs, including manual chicken sexing, in order to sort chicks because the laying hen cross from which the eggs are generated usually will not give rise to male offspring.

### Definitions

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, the preferred materials and methods are described herein. In describing and claiming the present invention, the following terminology will be used.

It is also to be understood that the terminology used herein is for the purpose of describing particular embodiments only, and is not intended to be limiting.

The articles “a” and “an” are used herein to refer to one or to more than one (i.e., to at least one) of the grammatical object of the article. By way of example, “an element” means one element or more than one element.

The term “about” and its grammatical equivalents in relation to a reference numerical value and its grammatical equivalents as used herein can include a range of values plus or minus 10% from that value. For example, the amount “about 10” includes amounts from 9 to 11. The term “about” in relation to a reference numerical value can also include a range of values plus or minus 10%, 9%, 8%, 7%, 6%, 5%, 4%, 3%, 2%, or 1% from that value.

As used herein, the terms “allosomes” or “allosome” refer to chromosome that determine gender of an offspring. Allosomes are sometimes referred to as sex chromosomes.

### Animal Genetics and Genotypes

The two categories of chromosomes are autosomes and allosomes (sex chromosomes). Autosomes are other chromosomes that are not allosomes. The allosomes carry the genetic material that determines the gender of an offspring. In mammals, such as humans, cows, or bovines, males are the heterogametic sex which means they have two different sex chromosomes X and Y. The mammalian Y chromosome is a crucial factor for determining gender in mammals. In this case, the female is determined by XX and the male is XY. However, in poultry species and reptiles, such as chickens, females are the heterogametic sex. The allosomes are referred to as Z and W. The female W chromosome in this case is instead an important factor for sex determination. The female chicken has the allosomes ZW while the male chicken has the allosomes ZZ. In male offspring, one of the Z chromosomes is derived from the male parent, while the other Z chromosome is derived from the female parent.

As used herein, Z¹ is used to describe a Z derived from the female parent (hen), and Z² is used to describe a Z derived from the male (rooster).

Chromosomes and genes come in pairs, and each parent contributes one gene in each pair of genes. If two copies of the genes are the same, the genotype or genetic state is referred to as homozygous. However, if two copies of the genes are different, the genotype in this case is referred to as heterozygous.

In some instances, there are two methods to genetically modify chickens such that a single sex offspring is produced. The first method results in offspring that remains genetically modified in a detectable way, and the other produces chickens that are indistinguishable from wildtype specimens. In either case, the unfertilized egg sold for consumption should be indistinguishable from wildtype as they lack viable cellular material.

CRISPR based approaches can be employed to affect single sex offspring, but because they require the parental birds to express an active CRISPR nuclease, they can result in chromosomal aberrations. These characteristics are undesirable. There is also evidence that birds expressing CRISPR proteins may not grow as well or be as fertile as birds that do not express CRISPR.

In some instances, for genetically modified offspring, the chicken will ideally express the heterologous protein very early in embryogenesis and not thereafter such that its impact, if any is minimized.

The present disclosure provides methods and compositions whereby eggs that would otherwise bear male chickens are suppressed by utilizing protein splicing mechanism that involves split inteins system or by utilizing promoter driven toxin under specific transcription factor. In some instances, the present disclosure provides methods and compositions whereby eggs that would otherwise bear female chickens are suppressed by utilizing protein splicing mechanism that involves split inteins system.

### Intein-Mediated Protein Splicing to Generate Single Sex Offspring

Protein splicing is a post-translational mechanism by which an intein or internal protein fragment catalyzes its own excision from a precursor protein by joining the N-terminal extein (N-extein) and C-terminal extein (C-extein) where exteins are non-intein proteins adjacent to the intein. As a result of splicing, the resulting protein or final product, referred to as extein, contains the N-extein linked to the C-extein.

Intein-mediated protein splicing was first identified in bacteria and has been reported in other organisms (e.g., fungi and lower plants). Intein-mediated protein splicing does not require an energy supply, exogenous proteases, or cofactors. Splice junction proximal residues and internal residues within the intein direct these reactions. In some instances, a properly folded structure of the expressed protein facilitates efficient protein splicing. In some instances, certain peptide sequences surrounding the ligation junction (at N-extein and C-extein) are important for efficient trans-splicing to occur. In some aspects, for efficient trans-splicing, the first residue (C+1) in the C-extein can be an amino acid containing a thiol or hydroxyl group (e.g., Cys, Ser, or Thr). In some aspects, residue 1 or the intein can be Cys or Ser. (See Topilina, N. et al. 2014. Mobile DNA. 5:5; Tornabene, P. et al. 2019. Sci Trans Med.).

In some instances, the mechanism of protein splicing involves four main steps. In the first step, which is called amide-thioester rearrangement, the peptide bond linking the N-extein and intein is converted to a thioester or ester via nucleophilic attack by the N-terminal Cys or Ser of the intein. In the next step, which is also known as transesterification, the N-extein is transferred from the side chain of the first intein residue to the side chain of the first X-extein residue, which results in a branched ester intermediate. During the third step, the branched ester is resolved by Asn cyclization coupled to peptide bond cleavage. This step is also known as Asparagine cyclization, and this leaves the ligated exteins which are linked by ester bond separated from the intein. At this stage, the intein has a C-terminal aminosuccinimide. In the last step, which is also the finishing reaction, the ester bond connecting the ligated exteins is rapidly converted to the amide bond, resulting in a new polypeptide. In some instances, the C-terminal aminosuccinimide of the intein may be hydrolyzed. In some instances, the ester bond involved in protein splicing can be a thioester bond. (See Topilina, N. et al. 2014. Mobile DNA. 5:5).

As used herein, the terms “inteins” or “intein” refer to internal protein fragment or intervening protein fragment that will be cleaved off during protein splicing and is not part of the final polypeptide. In some instances, an intein is a segment of protein that is able to excise itself. In some instances, the inteins are present when the protein is first made, but are later spliced out and are not part of the final polypeptide.

As used herein, the terms “exteins” or “extein” refer to protein fragments or remaining portions of the protein fragments that will be joined by a peptide bond to form the final polypeptide as a result of protein splicing.

### Split Inteins System

Split inteins are a subset of inteins that are expressed as two separate polypeptides at the ends of two proteins which catalyze their trans-splicing resulting in a single larger polypeptide (See Tornabene, P. et al. 2019. Sci Trans Med.). Split inteins are also known as protein trans-splicing (PTS) or trans-splicing inteins. In split inteins system, the two fragments of inteins are transcribed and translated by two independent genes and requires co-expression of both split intein fragments: N-intein (or N-terminal split intein) and C-intein (or C-terminal split intein). The two split intein fragments are capable of reconstituting splicing activity in much the same way as if they were synthesized as a single contiguous polypeptide and catalyze the ligation of N-extein and C-extein. (See Wang, H. et al. 2022. Frontiers in Bioengineering and Biotechnology).

Overall, inteins can seamlessly excise themselves from larger proteins (See Gogarten, J. et al. 2002. Annu Rev Microbiol. 56:263-87) and inteins could be expressed in fragments such that the fragments retained the ability to effect splicing in trans (See Aranko, A. et al. 2014. Protein Eng Des Sel. (8):263-71). The size of the trans complementing inteins can be reduced, providing an effective tool for protein engineering (See Lew, B. et al. 1998. J Biol Chem. (26):15887-90).

In some instances, the present disclosure provides methods and compositions to generate single sex offspring, e.g., female hens, by utilizing split inteins system. In some instances, one toxin fragment is fused with N-terminal split intein in one expression cassette and the other toxin fragment is fused with C-terminal split intein in another expression cassette, whereby when the offspring, e.g., male chicken, harbors both expression cassettes, the two toxin fragments join via trans-splicing inteins, thereby generating a functional toxin that kills the offspring.

In some instances, split inteins ligate the exteins via a peptide bond. In some instances, short native extein sequences are added to optimize the split site.

In some embodiments, the inteins are expressed as two separate fragments. In some embodiments, the two separate fragments of inteins are expressed natively. In some embodiments, the two separate fragments of inteins are expressed by protein engineering. In some embodiments, the expression of the two separate fragments of inteins facilitates the protein splicing. In some embodiments, the protein splicing occurs in trans (protein trans-splicing or PTS). In some embodiments, reassociation of the two separate intein fragments occurs prior to the protein splicing. In some embodiments, the intein fragments are split intein fragments.

In some embodiments, the inteins are full-length inteins. In some embodiments, the inteins are mini-inteins. In some embodiments, the inteins are split inteins. In some embodiments, the split inteins are naturally occurring inteins. In some embodiments, the split inteins are synthetic inteins.

### Expression Cassette for Split Inteins System

Provided herein are expression cassettes comprising nucleotide sequences encoding a transgene or gene of interest and regulatory sequence to be expressed by a transfected cell. In some embodiments, one or more expression cassettes are used to generate engineered animal. In some embodiments, the engineered animal includes, but not limited to, cow, mouse, rat, rabbit, guinea pig, chicken, fish, bird, reptile, camelid, bovine, chimpanzee, sheep, goat, and non-human primate.

As used herein, the terms “integration site” or “integrate” refer to the DNA constructs or vectors carrying one or more expression cassette(s) that are integrated into the chromosome in such a way that they are expressed and do not cause health issues for animal. In some instances, the one or more expression cassette(s) is integrated into one chromosome. In some instances, the one or more expression cassette(s) is integrated into both chromosomes. In some instances, the chromosome in which the one or more expression cassette(s) is integrated into is an allosome. In some instances, the chromosome in which the one or more expression cassette(s) is integrated into is an autosome.

In some embodiments, the one or more expression cassette(s) is integrated into a chromosome. In some embodiments, the chromosome is an autosome. In some embodiments, the chromosome is an allosome. In some embodiments, the one or more expression cassette(s) is integrated into both chromosomes. In some embodiments, the chromosomes are autosomes.

As used herein, the terms “transgene” or “gene of interest” are used interchangeably to refer to a nucleotide sequence containing a gene sequence that has been isolated from one organism and is introduced into a different organism. In some instances, the transgene refers to an exogenous gene that is introduced into a cell or an organism by genetic engineering techniques. In some instances, the transgene is transferred into the target cell via a vector or expression cassette.

In some instances, the one or more expression cassette(s) carry a transgene or gene of interest. In some instances, the transgene or gene of interest comprises protein-coding genes. In some instances, the protein-coding genes encode a toxin or toxic protein. In some instances, the protein-coding genes encode a toxin fragment. In some instances, the protein-coding genes encode a disease resistant protein. In some instances, a transgene or gene of interest comprises an engineered protein. In some embodiments, the engineered protein is a fusion protein. In some embodiments, the transgene or gene of interest comprises a full-length protein. In some embodiments, the transgene or gene of interest comprises a protein fragment. In some embodiments, the transgene or gene of interest comprises an active protein. In some embodiments, the transgene or gene of interest comprises an inactive protein or protein fragment. In some embodiments, the transgene or gene of interest comprises an inactive protein or protein fragment fused to an intein. In some embodiments, the intein is a C-intein. In some embodiments, the intein is a C-terminal split intein. In some embodiments, the intein is a N-intein. In some embodiments, the N-intein is a N-terminal split intein. In some embodiments, the inactive protein or protein fragment fused to the inteins are activated or become functional protein via split inteins system.

In some embodiments, the first expression cassette carries the one or more nucleotide sequence encoding the first split intein fragment fused to the first toxin fragment. In some embodiments, the second expression cassette carries the one or more nucleotide sequence encoding the second split intein fragment fused to the second toxin fragment. In some embodiments, the one or more nucleotide sequences are integrated in one or more chromosomes in the non-human vertebrate animal. In some embodiments, the one or more nucleotide sequences are carried by one or more expression cassettes or vectors. In some embodiments, the chromosome is an autosome. In some embodiments, the chromosome is an allosome.

In some embodiments, the one or more nucleotide sequences encode a split intein fragment fused to a toxin fragment.

In some embodiments, the first split intein fragment is fused to the first toxin fragment. In some embodiments, the first split intein fragment comprises an N-terminal split intein. In some embodiments, the first split intein fragment comprises a C-terminal split intein. In some embodiments, the first toxin fragment comprises an N-terminal toxin. In some embodiments, the first toxin fragment comprises a C-terminal toxin.

In some embodiments, the second split intein fragment is fused to the second toxin fragment. In some embodiments, the second split intein fragment comprises an N-terminal split intein. In some embodiments, the second split intein fragment comprises a C-terminal split intein. In some embodiments, the second toxin fragment comprises an N-terminal toxin. In some embodiments, the second toxin fragment comprises a C-terminal toxin.

As used herein, the terms “promoter” refers to a section of DNA to which proteins, e.g., transcription factors, bind and induce transcription of the adjacent gene located downstream of the promoter. In some instances, promoters are more active or less active, e.g., driving more transcription or less transcription of the downstream gene either based on their intrinsic strength as a promoter or in response to various signaling events. In some instances, promoters are active at certain times during development, e.g., during embryogenesis or early development. In some instances, promoters are active in certain cell type, e.g., hematopoietic progenitor cells. In some instances, proteins, e.g., transcription factors, can be conditionally recruited to a promoter region to increase transcription or decrease the transcription of the downstream gene.

In some embodiments, the one or more nucleotide sequences in the first non-human vertebrate animal further comprises a promoter. In some embodiments, the promoter is normally inactive in the adult non-human vertebrate animal. In some embodiments, the promoter is active during embryogenesis. In some embodiments, the promoter is active during embryogenesis and is silent or suppressed after embryogenesis. In some embodiments, the promoter is activated by a transcription factor. In some embodiments, the transcription factor comprises a small molecule. In some embodiments, the small molecule comprises a tetracycline compound.

In some embodiments, the one or more nucleotide sequences in the second non-human vertebrate animal further comprises a promoter. In some embodiments, the promoter is normally inactive in the adult non-human vertebrate animal. In some embodiments, the promoter is active during embryogenesis. In some embodiments, the promoter is active during embryogenesis and is silent or suppressed after embryogenesis. In some embodiments, the promoter is activated by a transcription factor.

In some embodiments, the promoter is normally active in the adult non-human vertebrate animal. In some embodiments, the promoter is active during embryogenesis. In some embodiments, the promoter is active in a wide range of cell types. In some embodiments, the promoter is active in a specific cell type.

In some embodiments, the promoter is a constitutive promoter, e.g., ovalbumin gene promoter, chicken β-actin, cytomegalovirus (CMV) enhancer (CCAG or CAG promoter), histone H4 promoter, phosphoglycerol kinase (PGK) promoter, or other constitutive promoters. In some embodiments, the promoter is an inducible promoter system, e.g., temperature-inducible gene regulation (TIGR system) or tetracycline-controlled inducible operator system.

In some embodiments, the first promoter is normally inactive in the non-human vertebrate animal. In some embodiments, the first promoter is active during embryogenesis. In some embodiments, the first promoter is active during embryogenesis and is silent or suppressed after embryogenesis. In some embodiments, the second promoter is normally inactive in the non-human vertebrate animal. In some embodiments, the second promoter is active during embryogenesis. In some embodiments, the second promoter is active during embryogenesis and is silent or suppressed after embryogenesis.

As used herein, the terms “toxin” or “toxic protein” refer to any protein that is capable of killing or severely impairing the function of a cell. In some instances, the toxin is expressed as fragments and is not functional until the protein fragments bind to form an active or functional protein. In some instances, toxin fragment is fused with split inteins and is encoded by the one or more expression cassette(s). In some instances, the cell expressing functional toxin is lethal. For example, nuclease Barnase is bacterial protein that has ribonuclease activity. Nuclease Barnase can be toxic and is lethal to the cell when expressed without its inhibitor, Barstar. Split intein system have been used to reconstitute the nuclease barnase in several systems, most notably in plants. Upon reconstitution of the toxin due to co-expression of two split inteins and two toxin fragments, this toxin is in its active form and kills the cells (See Kempe, K. et al. 2009. Plant Biotechnol J. 7(3):283-97).

In some embodiments, the toxin includes, but not limited to, a nuclease, a ribosome toxin, or a protease. In some embodiments, the nuclease comprises Barnase, RNAse, or restriction endonucleases. In some embodiments, the ribosome toxin comprises diphtheria, ricin, abrin, or pokeweed antiviral protein. In some embodiments, the protease comprises caspases, proteinase K, trypsin, chymotrypsin, or papain. Other toxins capable of killing the host cell or endogenous protein whose overexpression is cytotoxic may be used.

In some embodiments, the toxin fragment comprises an N-terminal toxin. In some embodiments, the toxin fragment comprises a C-terminal toxin. In some embodiments, the N-terminal toxin is fused to the N-terminal split intein. In some embodiments, the N-terminal toxin is fused to the C-terminal split intein. In some embodiments, the C-terminal toxin is fused to the N-terminal split intein. In some embodiments, the C-terminal toxin is fused to the C-terminal split intein.

As used herein, the term “transcription terminator” or “terminator sequence” refer to a region of nucleic acid sequence that marks the end of a gene during transcription. In some instances, this region mediates transcriptional termination by triggering the release of transcript RNA from the translational complex. In some instances, the transcription terminator involves direct activity of termination factors. In some instances, the transcription terminator involves indirect activity of termination factors.

In some embodiments, the expression cassette in the first non-human vertebrate animal further comprises a transcription terminator. In some embodiments, the expression cassette in the second non-human vertebrate animal further comprises a transcription terminator. In some embodiments, the transcription terminator comprises poly-A signals. In some embodiments, the terminator sequences comprise sequence motif AAUAAA. In some embodiments, the terminator sequences comprise mammalian terminators, e.g., SV40, hGH, BGH, and rbGlob. Other terminator sequences or motifs can also be used.

As used herein, the term “intron” refers to a section of pre-mRNA that is removed via splicing and is not encoded in the translated protein. In some aspects, the intron encodes sequences that facilitate gene expression.

In some embodiments, the one or more nucleotide sequences further comprise an intron. In some aspects, the intron encodes sequences that facilitate the gene expression. In some embodiments, the intron is a naturally occurred intron encoded in the gene. In some embodiments, the intron is an engineered intron. In some embodiments, the engineered intron is placed at the 5′ end of the open reading frame of the DNA construct. In some embodiments, the intron is placed at the 3′ end of the mRNA to increase mRNA stability. In some embodiments, the intron comprises an AU-rich element that is placed at the 3′ end of the mRNA.

In some embodiments, the one or more nucleotide sequences comprises exons encoding a toxin or fragment thereof fused to an intein. In some embodiments, the toxin or fragment thereof is an active toxin. In some embodiments, the toxin or fragment thereof is an inactive toxin or toxin fragment. In some embodiments, the toxin or fragment thereof is an inactive toxin or toxin fragment fused to an intein. In some embodiments, the intein is a C-intein. In some embodiments, the intein is a C-terminal split intein. In some embodiments, the intein is a N-intein. In some embodiments, the N-intein is a N-terminal split intein. In some embodiments, the inactive toxin or toxin fragments thereof fused to the inteins are activated or become functional protein via split inteins system.

Delivery of the DNA constructs carrying one or more expression cassette(s) to generate engineered animal, e.g., chicken, is performed by viral transfection system, e.g., lentiviral based system. Alternatively, non-viral method is utilized. In some cases, the DNA constructs are delivered using CRISPR. The non-viral method is based on genetically modified embryonic cells carrying DNA construct to be transferred into the recipient embryo, thereby generating transgenic/engineered animal, e.g., chicken (see Bednarczyk, M. et al. 2018. 59:81-89).

In some embodiments, the method to generate engineered animal, e.g., chicken, comprises viral transfection system. In some embodiments, the viral transfection system is a lentiviral based system. In various embodiments, the method to generate engineered animal, e.g., chicken, comprises non-viral method, e.g., electroporation, lipofection, or CRISPR to transfer DNA construct into the targeted cell.

### Animals for Generation of Single Sex Offspring Via Split Inteins System

In one aspect, there are provided non-human vertebrate animals comprising one or more nucleotide sequences. In some embodiments, the one or more nucleotide sequences comprise a promoter, a split intein fragment, and a toxin fragment, and wherein the one or more nucleotide sequences are integrated in one or more chromosomes. In some embodiments, the one or more chromosomes is an allosome. In some embodiments, the one or more chromosomes is an autosome. In some embodiments, the split intein fragment is fused to the toxin fragment. In some embodiments, the toxin fragment is a toxin selected from the group consisting of a nuclease, a ribosome toxin, and a protease. In some embodiments, the nuclease comprises Barnase, an RNase, or a restriction endonuclease. In some embodiments, the ribosome toxin comprises diphtheria, ricin, abrin, or pokeweed antiviral protein. In some embodiments, the protease comprises a caspase, proteinase K, trypsin, chymotrypsin, or papain. In some embodiments, the non-human vertebrate animal is selected from the group consisting of cow, mouse, rat, rabbit, guinea pig, chicken, fish, bird, reptile, camelid, bovine, chimpanzee, sheep, goat, and non-human primate.

In another aspect, there are provided non-human vertebrate animals comprising one or more nucleotide sequences, wherein the one or more nucleotide sequences comprises a promoter, a first split intein fragment, a first toxin fragment, or any combination thereof. In some embodiments, the one or more nucleotide sequences further comprises an intron. In some embodiments, the one or more nucleotide sequences are integrated in one or more chromosomes in the non-human vertebrate animal. In some embodiments, the chromosome is an autosome. In some embodiments, the chromosome is an allosome. In some embodiments, the first split intein fragment is fused to the first toxin fragment. In some embodiments, the first split intein fragment comprises an N-terminal intein. In some embodiments, the first split intein fragment comprises a C-terminal intein. In some embodiments, the first toxin fragment comprises an N-terminal toxin. In some embodiments, the first toxin fragment comprises a C-terminal toxin. In some embodiments, the first toxin fragment is a toxin selected from the group consisting of a nuclease, a ribosome toxin, and a protease. In some embodiments, the nuclease comprises Barnase, an RNase, or a restriction endonuclease. In some embodiments, the ribosome toxin comprises diphtheria, ricin, abrin, or pokeweed antiviral protein. In some embodiments, the protease comprises a caspase, proteinase K, trypsin, chymotrypsin, or papain. In some embodiments, the promoter is normally inactive in an adult of the non-human vertebrate animal. In some embodiments, the promoter is active during embryogenesis.

In another aspect, there are provided non-human vertebrate animals comprising one or more nucleotide sequences, wherein the one or more nucleotide sequences comprises a promoter, a second split intein fragment, a second toxin fragment, or any combination thereof. In some embodiments, the one or more nucleotide sequences further comprise an intron. In some embodiments, the one or more nucleotide sequences are integrated in one or more chromosomes in the non-human vertebrate animal. In some embodiments, the chromosome is an autosome. In some embodiments, the chromosome is an allosome. In some embodiments, the second split intein fragment is fused to the second toxin fragment. In some embodiments, the second split intein fragment comprises an N-terminal intein. In some embodiments, the second split intein fragment comprises a C-terminal intein. In some embodiments, the second toxin fragment comprises an N-terminal toxin. In some embodiments, the second toxin fragment comprises a C-terminal toxin. In some embodiments, the second toxin fragment is a toxin is selected from the group consisting of a nuclease, a ribosome toxin, and a protease. In some embodiments, the nuclease comprises Barnase, an RNase, or a restriction endonuclease. In some embodiments, the ribosome toxin comprises diphtheria, ricin, abrin, or pokeweed antiviral protein. In some embodiments, the protease comprises a caspase, proteinase K, trypsin, chymotrypsin, or papain.

In a further aspect, there are provided pluralities of non-human vertebrate animals each comprising one or more nucleotide sequences integrated in one or more chromosomes. In some embodiments, a first non-human vertebrate animal comprises a first split intein fragment and a first toxin fragment. In some embodiments, a second non-human vertebrate animal comprises a second split intein fragment, and a second toxin fragment. In some embodiments, the chromosome is an autosome. In some embodiments, the chromosome is an allosome. In some embodiments, the first split intein fragment comprises an N-terminal intein. In some embodiments, the first split intein fragment comprises a C-terminal intein. In some embodiments, the first toxin fragment comprises an N-terminal toxin. In some embodiments, the first toxin fragment comprises a C-terminal toxin. In some embodiments, the second split intein fragment comprises an N-terminal intein. In some embodiments, the second split intein fragment comprises a C-terminal intein. In some embodiments, the second toxin fragment comprises an N-terminal toxin. In some embodiments, the second toxin fragment comprises a C-terminal toxin. In some embodiments, the first toxin fragment and/or the second toxin fragment is a toxin is selected from the group consisting of a nuclease, a ribosome toxin, and a protease. In some embodiments, the nuclease comprises Barnase, an RNase, or a restriction endonuclease. In some embodiments, the ribosome toxin comprises diphtheria, ricin, abrin, or pokeweed antiviral protein. In some embodiments, the protease comprises a caspase, proteinase K, trypsin, chymotrypsin, or papain. In some embodiments, the one or more nucleotide sequences in the first non-human vertebrate animal further comprises a promoter. In some embodiments, the promoter is normally inactive in the adult non-human vertebrate animal. In some embodiments, the promoter is active during embryogenesis. In some embodiments, the promoter is activated by a transcription factor.

In another aspect, there are provided non-human vertebrate animals comprising one or more nucleotide sequences, wherein the one or more nucleotide sequences comprises a promoter and a toxin or a fragment thereof, wherein the promoter is activated upon binding of a transcription factor. In some embodiments, the one or more nucleotide sequences further comprise an intron. In some embodiments, the toxin or the fragment thereof is a toxin selected from the group consisting of a nuclease, a ribosome toxin, and a protease. In some embodiments, the nuclease comprises Barnase, an RNase, or a restriction endonuclease. In some embodiments, the ribosome toxin comprises diphtheria, ricin, abrin, or pokeweed antiviral protein. In some embodiments, the protease comprises a caspase, proteinase K, trypsin, chymotrypsin, or papain. In some embodiments, the one or more nucleotide sequences are integrated in one or more chromosomes in the non-human vertebrate animal. In some embodiments, the chromosome is an autosome. In some embodiments, the chromosome is an allosome.

In a further aspect, there are provided non-human vertebrate animals comprising one or more nucleotide sequences, wherein the one or more nucleotide sequences comprises a promoter and a transcription factor. In some embodiments, the one or more nucleotide sequences further comprise an intron. In some embodiments, the one or more nucleotide sequences are integrated in one or more chromosomes in the non-human vertebrate animal. In some embodiments, the chromosome is an autosome. In some embodiments, the chromosome is an allosome.

### Poultry

In one aspect, the present disclosure provides an engineered poultry or reptile, e.g., chickens, for generation of single sex offspring, e.g., female layer hens. In some instances, a female chicken in the first generation is engineered to harbor one or more expression cassette(s) encoding a toxin fragment fused with N-terminal split intein. This expression cassette is integrated into the female chicken chromosome. In some instances, the one or more expression cassette(s) is integrated into the Z allosome (called Z¹). In this instance, the genotype of the engineered female chicken is Z¹W. In this instance, two types of male chicken in parental generation are generated. The first type of male chicken is engineered to harbor the same expression cassette on both chromosomes as in the engineered female chicken. In some instances, both chromosomes in the first type of male chicken are Z allosomes, thus, the genotype of this male chicken is Z¹Z¹.

The second type of male chicken is engineered to harbor another expression cassette encoding another toxin fragment fused with C-terminal split intein, and, in some instances, this expression cassette is integrated into both chromosomes, e.g., both Z allosomes. In this instance, the genotype of the second type of engineered male chicken is Z²Z². The two toxin fragments encoding by Z¹ and Z² are joined by split inteins system, thereby generating a functional or active toxin that kills the cell that harbors both Z¹ and Z² expression cassettes.

In some instances, the Z¹ allosome is integrated with another expression cassette encoding a toxin fragment fused with C-terminal split intein, and in this instance, the Z² allosome is integrated with the other expression cassette encoding another toxin fragment fused with N-terminal split intein. The two toxin fragments encoding by Z¹ and Z² are joined by split inteins system, thereby generating a functional or active toxin that kills the cell that harbors both Z¹ and Z² expression cassettes.

In order to generate the second generation of chickens from the engineered female and male chickens, two different crosses may be employed. First, Z¹W female grandparent is crossed with Z²Z² male grandparent. This will generate viable Z²W female parent chicken because Z¹Z² male parent chicken expresses both expression cassettes and this generates functional or active toxin via a split inteins system. Thus, male chicks are not viable. In some instances, the Z²W female parent chicken is crossed with Z¹Z¹ male chicken, thereby generating Z¹W female offspring chicken because Z¹Z² male offspring expresses both expression cassettes and this generates functional or active toxin via split inteins system. Thus, generation of single sex offspring, e.g., female chicken is achieved via split inteins system. Crossing Z¹W with Z²Z² or Z²W with Z¹Z¹ will generate single sex offspring chicken, e.g., female chicken, as long as Z¹ and Z² encode different inteins (N-terminal split intein in one and C-terminal intein in another) that join to form functional or active toxin.

In some instances, an engineered chicken is generated for generation of single sex offspring in the offspring generation. In this instance, the female parent chicken is engineered to harbor one or more expression cassette(s) encoding a toxin fragment fused with N-terminal split intein. This expression cassette is integrated into the female chicken chromosome. In some instances, the one or more expression cassette(s) is integrated into the Z allosome (called Z¹). In this instance, the genotype of female parent chicken is Z¹W. In this instance, the male parent chicken is engineered to harbor another expression cassette encoding another toxin fragment fused with C-terminal split intein, and, in this instance, this expression cassette is integrated into both chromosomes, e.g., both Z allosomes. In this instance, the genotype of the male parent chicken is Z²Z². The two toxin fragments encoding by Z¹ and Z² are joined by split inteins system, thereby generating a functional or active toxin that kills the cell that harbors both Z¹ and Z² expression cassettes. In order to generate single sex offspring generation, the female parent Z¹W is crossed with the male parent Z²Z², thereby generating Z²W female offspring because Z¹Z² male offspring expresses both expression cassettes and this generates functional or active toxin via split inteins system, thus not viable.

In other instances, an engineered chicken is generated for generation of single sex offspring in the offspring generation. In this instance, the female parent chicken is engineered to harbor one or more expression cassette(s) encoding a toxin fragment fused with C-terminal split intein. This expression cassette is integrated into chicken chromosome. In some instances, the one or more expression cassette(s) is integrated into the Z allosome (called Z¹). In this instance, the genotype of the female parent chicken is Z¹W. In this instance, the male parent chicken is engineered to harbor another expression cassette encoding another toxin fragment fused with N-terminal split intein, and, in this instance, this expression cassette is integrated into both chromosomes, e.g., both Z allosomes. In this instance, the genotype of the male parent chicken is Z²Z². The two toxin fragments encoding by Z¹ and Z² are joined by split inteins system, thereby generating a functional or active toxin that kills the cell that harbors both Z¹ and Z² expression cassettes. In order to generate single sex offspring generation, the female parent Z¹W is crossed with the male parent Z²Z², thereby generating Z²W female offspring because Z¹Z² male offspring expresses both expression cassettes and this generates functional or active toxin via split inteins system, and the offspring thus are not viable. Crossing Z¹W with Z²Z² or Z²W with Z¹Z¹ will generate single sex offspring chicken, e.g., female chicken as long as Z¹ and Z² encode different inteins (N-terminal split intein in one and C-terminal split intein in another) that join to form functional or active toxin.

### Mammals

In another aspect, the present disclosure provides an engineered mammal, e.g., cows, for generation of single sex offspring, e.g., female cows. In some instances, a male cow in the first generation is engineered to harbor one or more expression cassette(s) encoding a toxin fragment fused with N-terminal split intein. This expression cassette is integrated into the male cow chromosome. In some instances, the one or more expression cassette(s) is integrated into the Y allosome (called Y¹). In this instance, the genotype of the engineered male cow is XY¹.

The female cow in parental generation is engineered to harbor another expression cassette encoding another toxin fragment fused with C-terminal split intein, and, in some instances, this expression cassette is integrated into both chromosomes, e.g., both X allosomes. In this instance, the genotype of the engineered female cow is X²X². The two toxin fragments encoding by Y¹ and X² are joined by split inteins system, thereby generating a functional or active toxin that kills the cell that harbors both Y¹ and X² expression cassettes.

In some instances, the Y¹ allosome is integrated with another expression cassette encoding a toxin fragment fused with C-terminal split intein, and in this instance, the X² allosome is integrated with the other expression cassette encoding another toxin fragment fused with N-terminal split intein. The two toxin fragments encoding by Y¹ and X² are joined by split inteins system, thereby generating a functional or active toxin that kills the cell that harbors both Y¹ and X² expression cassettes.

In order to generate the offspring generation of cows from the engineered female and male cows, X²X² female parent is crossed with XY¹ male parent. This will generate viable XX² female offspring cow because X²Y¹ male offspring expresses both expression cassettes and this generates functional or active toxin via a split inteins system. Thus, male cows are not viable. Generation of single sex offspring, e.g., female cow, is achieved via split inteins system. This trans-splicing intein system for generation of single sex offspring can be applied to other animal, all of which are compatible with methods of the present disclosure and contemplated herein. Examples of animal include, but not limited to mammals, e.g., cow, mouse, rat, rabbit, guinea pig, bovine, chimpanzee, sheep, goat, and non-human primate.

### Methods of Generation of Single Sex Offspring Via Split Inteins System

By crossing the engineered animal as described in the present disclosure, generation of single sex offspring can be achieved. In one aspect, provided herein are methods of producing a single sex population of non-human vertebrate animals. In some embodiments, the method comprises crossing a first non-human vertebrate animal having a first genotype comprising a pair of heterozygous chromosomes comprising one or more nucleotide sequences encoding a first toxin fragment with a second non-human vertebrate animal having a second genotype comprising a pair of homozygous chromosomes comprising one or more nucleotide sequences encoding a second toxin fragment; wherein a resulting progeny having a genotype comprising the first toxin fragment and the second toxin fragment is not viable; thereby creating a single sex population. In some embodiments, the chromosome is an autosome. In some embodiments, the chromosome is an allosome. In some embodiments, the non-human vertebrate animal is selected from the group consisting of cow, mouse, rat, rabbit, guinea pig, chicken, fish, bird, reptile, camelid, bovine, chimpanzee, sheep, goat, and non-human primate. In some embodiments, the first toxin fragment and/or the second toxin fragment is a toxin selected from the group consisting of a nuclease, a ribosome toxin, and a protease. In some embodiments, the nuclease comprises Barnase, an RNase, or a restriction endonuclease. In some embodiments, the ribosome toxin comprises diphtheria, ricin, abrin, or pokeweed antiviral protein. In some embodiments, the protease comprises a caspase, proteinase K, trypsin, chymotrypsin, or papain.

In another aspect, there are provided methods of producing a single sex population of non-human vertebrate animals, comprising the steps of: obtaining a first non-human vertebrate animal, wherein one or more chromosomes comprise a first expression cassette comprising the following elements in 5′ to 3′ orientation: a first promoter, operatively linked thereto a nucleotide sequence encoding for a first toxin fragment and a first split intein fragment; obtaining a second non-human vertebrate animal, wherein one or more allosomes comprise a second expression cassette comprising the following elements in 5′ to 3′ orientation: a second promoter, operatively linked thereto a nucleotide sequence encoding for a second toxin fragment and a second split intein fragment; crossing the first non-human vertebrate animal and the second non-human vertebrate animals, wherein a resulting progeny containing the first expression cassette and the second expression cassette is not viable; thereby creating a single sex population. In some embodiments, the chromosome is an autosome. In some embodiments, the chromosome is an allosome. In some embodiments, the first split intein fragment comprises an N-terminal intein. In some embodiments, the first split intein fragment comprises a C-terminal intein. In some embodiments, the first toxin fragment comprises an N-terminal toxin. In some embodiments, the first toxin fragment comprises a C-terminal toxin. In some embodiments, the second split intein fragment comprises an N-terminal intein. In some embodiments, the second split intein fragment comprises a C-terminal intein. In some embodiments, the second toxin fragment comprises an N-terminal toxin. In some embodiments, the second toxin fragment comprises a C-terminal toxin. In some embodiments, the first toxin fragment and/or the second toxin fragment is a toxin is selected from the group consisting of a nuclease, a ribosome toxin, and a protease. In some embodiments, the nuclease comprises Barnase, an RNase, or a restriction endonuclease. In some embodiments, the ribosome toxin comprises diphtheria, ricin, abrin, or pokeweed antiviral protein. In some embodiments, the protease comprises a caspase, proteinase K, trypsin, chymotrypsin, or papain. In some embodiments, the first promoter is normally inactive in the non-human vertebrate animal. In some embodiments, the first promoter is active during embryogenesis. In some embodiments, the expression cassette in the first non-human vertebrate animal further comprises a terminator sequence. In some embodiments, the expression cassette in the second non-human vertebrate animal further comprises a terminator sequence.

In a further aspect, there are provided processes of producing a non-human vertebrate animal or parts thereof expressing a trait of interest, the trait having a controlled distribution of the trait to progeny, thereby creating a single sex population, wherein the process comprises: obtaining a first non-human vertebrate animal or a cell thereof having in homologous chromosomes, a first nucleotide sequence comprising a first fragment of a nucleotide sequence encoding the trait of interest; obtaining a second non-human vertebrate animal or a cell thereof having in a second locus of the chromosome heterozygous to the chromosome of step (i), a second nucleotide sequence comprising a second fragment of the nucleotide sequence encoding the trait of interest; and crossing the first non-human vertebrate animals or cells thereof and the second non-human vertebrate animals or cells thereof to generate progeny exhibiting the trait of interest due to binding between a protein or polypeptide encoded by the first nucleotide sequence and a protein or polypeptide encoded by the second nucleotide sequence, wherein the genotype expressing both the first nucleotide sequence and second nucleotide sequence is not viable. In some embodiments, the chromosome is an autosome. In some embodiments, the chromosome is an allosome.

In another aspect, there are provided methods of producing a single sex population of non-human vertebrate animals, comprising the steps of: obtaining a first non-human vertebrate comprising one or more nucleotide sequences integrated in one or more chromosomes comprises the following elements in 5′ to 3′ orientation: a first inactive promoter, operatively linked thereto a nucleotide sequence encoding for a toxin; obtaining a second non-human vertebrate animal comprising one or more nucleotide sequences integrated in one or more chromosomes comprising the following elements in 5′ to 3′ orientation: a second promoter, operatively linked thereto a transcription factor corresponding to the inactive promoter; and crossing the first non-human vertebrate animal and the second non-human vertebrate animals, wherein a resulting progeny, containing the first inactive promoter, the second promoter expressing the transcription factor corresponding to the first inactive promoter, and the corresponding transcription factor, is not viable; thereby creating a single sex population. In some embodiments, the chromosome is an autosome. In some embodiments, the chromosome is an allosome. In some embodiments, the toxin is selected from the group consisting of a nuclease, a ribosome toxin, and a protease. In some embodiments, the nuclease comprises Barnase, an RNase, or a restriction endonuclease. In some embodiments, the ribosome toxin comprises diphtheria, ricin, abrin, or pokeweed antiviral protein. In some embodiments, the protease comprises a caspase, proteinase K, trypsin, chymotrypsin, or papain.

### Single Sex Offspring Generated Via Split Inteins System

In one aspect, provided herein are non-human vertebrate animals having a genotype comprising a pair of chromosomes, wherein the chromosome comprises a toxin fragment, wherein the toxin fragment is not functional. In some embodiments, the chromosome is an autosome. In some embodiments, the chromosome is an allosome. In some embodiments, the toxin fragment comprises an N-terminal toxin. In some embodiments, the toxin fragment comprises a C-terminal toxin. In some embodiments, the toxin fragment is a toxin selected from the group consisting of a nuclease, a ribosome toxin, and a protease. In some embodiments, the nuclease comprises Barnase, an RNase, or a restriction endonuclease. In some embodiments, the ribosome toxin comprises diphtheria, ricin, abrin, or pokeweed antiviral protein. In some embodiments, the protease comprises a caspase, proteinase K, trypsin, chymotrypsin, or papain.

In another aspect, there are provided non-human vertebrate animals having a genotype comprising a pair of heterozygous chromosomes, wherein the chromosome comprises a promoter and a toxin or a fragment thereof, wherein the promoter is activated upon binding of a transcription factor. In some embodiments, the chromosome is an autosome. In some embodiments, the chromosome is an allosome. In some embodiments, the toxin or the fragment thereof is a toxin selected from the group consisting of a nuclease, a ribosome toxin, and a protease. In some embodiments, the nuclease comprises Barnase, an RNase, or a restriction endonuclease. In some embodiments, the ribosome toxin comprises diphtheria, ricin, abrin, or pokeweed antiviral protein. In some embodiments, the protease comprises a caspase, proteinase K, trypsin, chymotrypsin, or papain.

### Single Sex Offspring in Poultry

In one aspect, the present disclosure provides methods and compositions utilizing a trans-splicing intein system that is sufficiently small to accomplish the task of toxin reconstruction to generate single sex offspring in animal such as chicken. In some instances, the single sex offspring is female offspring. For example, in chicken, the Z¹ that is derived from the female parent (hen) can have the C-terminal split intein linked to the toxin fragment, and the Z² which is derived from the male (rooster) can have the N-terminal split intein linked to the other half of the toxin fragment in such a way that the toxin is functional when the two toxin fragments are combined. As a result of crossing between these two parents and a trans-splicing intein, male offspring have functional toxin that kills cells, allowing female offspring to survive. This trans-splicing intein system for generation of single sex offspring can be applied to other animal, all of which are compatible with methods of the present disclosure and contemplated herein. Examples of animal include, but not limited to chicken, bird, and reptile.

In different aspect, the present disclosure provides methods and compositions utilizing a trans-splicing intein system that is sufficiently small to accomplish the task of toxin reconstruction to generate single sex offspring in animal such as chicken. In some instances, the single sex offspring is a female offspring. For example, the male parent can have the C-terminal split intein linked to the toxin fragment. This C-terminal split intein-toxin fragment can be integrated into both autosomes. The female parent can have the N-terminal split intein linked to another toxin fragment integrated into a Z allosome. Thus, as a result of crossing between these two parents and a trans-splicing intein, the male offspring have functional toxin that kills cell, and leave female offspring viable. This trans-splicing intein system for generation of single sex offspring can be applied to other animal, all of which are compatible with methods of the present disclosure and contemplated herein. Examples of animal include, but not limited to chicken, bird, and reptile. In some embodiments, the male parent harbors N-terminal split intein linked to the toxin fragment and the female parent harbors C-terminal split intein linked to another toxin fragment.

In different aspect, the present disclosure provides methods and compositions utilizing a trans-splicing intein system that is sufficiently small to accomplish the task of toxin reconstruction to generate single sex offspring in animal such as chicken. In some instances, the single sex offspring is male offspring. For example, the male parent can have the C-terminal split intein linked to the toxin fragment. This C-terminal split intein-toxin fragment can be integrated into both autosomes. The female parent can have the N-terminal split intein linked to another toxin fragment integrated into a W allosome. Thus, as a result of crossing between these two parents and a trans-splicing intein, the female offspring have functional toxin that kills cell, and leave male offspring viable. This trans-splicing intein system for generation of single sex offspring can be applied to other animal, all of which are compatible with methods of the present disclosure and contemplated herein. Examples of animal include, but not limited to chicken, bird, and reptile. In some embodiments, the male parent harbors N-terminal split intein linked to the toxin fragment and the female parent harbors C-terminal split intein linked to another toxin fragment.

### Single Sex Offspring in Mammals

In one aspect, the present disclosure provides methods and compositions utilizing a trans-splicing intein system that is sufficiently small to accomplish the task of toxin reconstruction to generate single sex offspring in animal such as cows or pigs. In some instances, the single sex offspring is female offspring. For example, the male parent can have the C-terminal split intein linked to the toxin fragment. This C-terminal split intein-toxin fragment can be integrated into an Y allosome. The female parent can have the N-terminal split intein linked to another toxin fragment integrated into both autosomes. Thus, as a result of crossing between these two parents and a trans-splicing intein, the male offspring have functional toxin that kills cell, and leave female offspring viable. This trans-splicing intein system for generation of single sex offspring can be applied to other animal, all of which are compatible with methods of the present disclosure and contemplated herein. Examples of animal include, but not limited to mammals, e.g., cow, mouse, rat, rabbit, guinea pig, bovine, chimpanzee, sheep, goat, and non-human primate. In some embodiments, the male parent harbors an N-terminal split intein linked to the toxin fragment and the female parent harbors an C-terminal split intein linked to another toxin fragment.

In another aspect, the present disclosure provides methods and compositions utilizing a trans-splicing intein system that is sufficiently small to accomplish the task of toxin reconstruction to generate single sex offspring in animal such as cows or pigs. In some instances, the single sex offspring is male offspring. For example, the male parent can have the C-terminal split intein linked to the toxin fragment. This C-terminal split intein-toxin fragment can be integrated into an X allosome. The female parent can have the N-terminal split intein linked to another toxin fragment integrated into both autosomes. Thus, as a result of crossing between these two parents and a trans-splicing intein, the female offspring have functional toxin that kills cell, and leave male offspring viable. This trans-splicing intein system for generation of single sex offspring can be applied to other animal, all of which are compatible with methods of the present disclosure and contemplated herein. Examples of animal include, but not limited to mammals, e.g., cow, mouse, rat, rabbit, guinea pig, bovine, chimpanzee, sheep, goat, and non-human primate. In some embodiments, the male parent harbors N-terminal split intein linked to the toxin fragment and the female parent harbors C-terminal split intein linked to another toxin fragment.

### Expression Cassette for Promoter Driven Toxin Under Specific Transcription Factor

Provided herein are expression cassettes comprising nucleotide sequences encoding a transgene or gene of interest and regulatory sequence to be expressed by a transfected cell. In some embodiments, one or more expression cassettes are used to generate engineered animal. In some embodiments, the engineered animal includes, but not limited to, cow, mouse, rat, rabbit, guinea pig, chicken, fish, bird, reptile, camelid, bovine, chimpanzee, sheep, goat, and non-human primate.

As used herein, the terms “integration site” or “integrate” refer to the DNA constructs or vectors carrying one or more expression cassette(s) that are integrated into the chromosome in such a way that they are expressed and do not cause health issues for animal. In some instances, the one or more expression cassette(s) is integrated into one chromosome. In some instances, the one or more expression cassette(s) is integrated into both chromosomes. In some instances, the chromosome in which the one or more expression cassette(s) is integrated into is an allosome. In some instances, the chromosome in which the one or more expression cassette(s) is integrated into is an autosome.

In some embodiments, the one or more expression cassette(s) is integrated into a chromosome. In some embodiments, the chromosome is an autosome. In some embodiments, the chromosome is an allosome. In some embodiments, the one or more expression cassette(s) is integrated into both chromosomes. In some embodiments, the chromosomes are autosomes.

In some embodiments, one or more expression cassettes carry one or more nucleotide sequences. In some embodiments, the one or more nucleotide sequences encode an inactive promoter and a toxin. In some embodiments, the one or more nucleotide sequences encode a specific transcription factor that can activate the inactive promoter. In some embodiments, the one or more nucleotide sequences are integrated in one or more chromosomes in the non-human vertebrate animal. In some embodiments, the one or more nucleotide sequences are carried by one or more expression cassettes or vectors. In some embodiments, the chromosome is an autosome. In some embodiments, the chromosome is an allosome. In some embodiments, the one or more nucleotide sequences encode a promoter that is conditionally activated by a specific transcription factor and a toxin. In some embodiments, the one or more nucleotide sequences encode the specific transcription factor to activate the promoter.

As used herein, the terms “promoter” refers to a section of DNA to which proteins, e.g., transcription factors, bind and induce transcription of the adjacent gene located downstream of the promoter. In this instance, the promoter is inactive and active upon binding of specific transcription factor. In some embodiments, the promoter is inducible promoter. In some embodiments, the promoter is activated by a transcription factor. In some embodiments, the transcription factor comprises a small molecule. In some embodiments, the small molecule comprises a tetracycline compound.

In some embodiments, the promoter is an inducible promoter system, e.g., temperature-inducible gene regulation (TIGR system) or tetracycline-controlled inducible operator system.

In some embodiments, the first expression cassette carries the one or more nucleotide sequence encoding the promoter that is activated by the specific transcription factor and a transgene or gene of interest. In some embodiments, the second expression cassette carried the one or more nucleotide sequence encoding the specific transcription factor to active the promoter. In some embodiments, the one or more expression cassette(s) is integrated into a chromosome. In some embodiments, the chromosome is an autosome. In some embodiments, the chromosome is an allosome. In some embodiments, the one or more expression cassette(s) is integrated into both chromosomes. In some embodiments, the chromosomes are autosomes.

In some instances, the transgene or gene of interest comprises protein-coding genes. In some instances, the protein-coding genes encode toxin or toxic protein. In some instances, the protein-coding genes encode antibiotic protein. In some instances, the protein-coding genes encode disease resistant protein. In some instances, a transgene or gene of interest comprises an engineered protein. In some embodiments, the engineered protein is a fusion protein. In some embodiments, the transgene or gene of interest comprises a full-length protein. In some embodiments, the transgene or gene of interest comprises an active protein. In some embodiments, the transgene or gene of interest comprises a functional or active toxin. In some instances, the transgene or gene of interest comprises a transcription factor.

As used herein, the terms “toxin” or “toxic protein” refer to any protein that is capable of killing or severely impairing the function of a cell. In this instance, the toxin is expressed as an active or functional protein, thus, the cell expresses functional toxin is lethal. For example, nuclease Barnase is bacterial protein that has ribonuclease activity. Nuclease Barnase can be toxin and is lethal to the cell when expressed without its inhibitor, Barstar.

In some embodiments, the toxin includes, but not limited to, nuclease, ribosome toxin, and protease. In some embodiments, the nuclease comprises Barnase, RNAse, or restriction endonucleases. In some embodiments, the ribosome toxin comprises diphtheria, ricin, abrin, or pokeweed antiviral protein. In some embodiments, the protease comprises caspases, proteinase K, trypsin, chymotrypsin, or papain. Other toxins capable of killing the host cell or endogenous protein whose overexpression is cytotoxic may be used.

As used herein, the term “transcription terminator” or “terminator sequence” refer to a region of nucleic acid sequence that marks the end of a gene during transcription. In some instances, this region mediates transcriptional termination by triggering the release of transcript RNA from the translational complex. In some instances, the transcription terminator involves direct activity of termination factors. In some instances, the transcription terminator involves indirect activity of termination factors.

In some embodiments, the one or more expression cassette in the first non-human vertebrate animal further comprises a transcription terminator. In some embodiments, the one or more expression cassette in the second non-human vertebrate animal further comprises a transcription terminator. In some embodiments, the transcription terminator comprises poly-A signals. In some embodiments, the terminator sequences comprise sequence motif AAUAAA. In some embodiments, the terminator sequences comprise mammalian terminators, e.g., SV40, hGH, BGH, and rbGlob. Other terminator sequences or motifs can also be used.

As used herein, the term “intron” refers to a section of pre-mRNA that is removed via splicing and is not encoded in the translated protein. In some aspects, the intron encodes sequences that facilitate the gene expression.

In some embodiments, the one or more nucleotide sequences further comprise an intron. In some aspects, the intron encodes sequences that facilitate the gene expression. In some embodiments, the intron is a naturally occurred intron encoded in the gene. In some embodiments, the intron is an engineered intron. In some embodiments, the engineered intron is placed at the 5′ end of the open reading frame of the DNA construct. In some embodiments, the intron is placed at the 3′ end of the mRNA to increase mRNA stability. In some embodiments, the intron comprises an AU-rich element that is placed at the 3′ end of the mRNA.

Delivery of the DNA constructs carrying the one or more expression cassette(s) to generate engineered animal, e.g., chicken, can be performed by viral transfection system, e.g., lentiviral based system. Alternatively, non-viral method can be utilized. The non-viral method is based on genetically modified embryonic cells carrying DNA construct to be transferred into the recipient embryo, thereby generating transgenic/engineered animal, e.g., chicken (see Bednarczyk, M. et al. 2018. 59:81-89).

In some embodiments, the method to generate engineered animal, e.g., chicken, comprises viral transfection system. In some embodiments, the viral transfection system is lentiviral based system. In various embodiments, the method to generate engineered animal, e.g., chicken, comprises non-viral method, e.g., electroporation or lipofection to transfer DNA construct into the targeted cell.

### Animals for Generation of Single Sex Offspring Via Promoter Driven Toxin Under Specific Transcription Factor

In one aspect, the present disclosure provides a non-human vertebrate animal comprising one or more nucleotide sequences, wherein the one or more nucleotide sequences comprises a promoter and a toxin or a fragment thereof, wherein the promoter is activated upon binding of a transcription factor. In some embodiments, the one or more nucleotide sequences are integrated in one or more chromosomes in the non-human vertebrate animal. In some embodiments, the chromosome is an autosome. In some embodiments, the chromosome is an allosome.

In another aspect, the present disclosure provides a non-human vertebrate animal having a genotype comprising a pair of heterozygous chromosomes, wherein the chromosome comprises a promoter and a toxin or a fragment thereof, wherein the promoter is activated upon binding of a transcription factor. In some embodiments, the chromosome is an autosome. In some embodiments, the chromosome is an allosome.

In another aspect, the present disclosure provides a non-human vertebrate animal comprising one or more nucleotide sequences, wherein the one or more nucleotide sequences comprises a promoter and a transcription factor.

### Poultry

Engineered poultry or reptile, e.g., chickens, are generated for generation of single sex offspring, e.g., female layer hens. In some instances, a female chicken in the first generation is engineered to harbor one or more expression cassette(s) encoding an inducible promoter and a toxin located downstream of the inducible promoter. This expression cassette is integrated into the female chicken chromosome. In some instances, the one or more expression cassette(s) is integrated into the Z allosome (called Z¹). In this instance, the genotype of the engineered female chicken is Z¹W. In this instance, two types of male chicken in parental generation are generated. The first type of male chicken is engineered to harbor the same expression cassette on both chromosomes as in the engineered female chicken. In some instances, both chromosomes in the first type of male chicken are Z allosomes, thus, the genotype of this male chicken is Z¹Z¹.

The second type of male chicken is engineered to harbor another expression cassette encoding a transcription factor that activates the inducible promoter to express the toxin, and, in this instance, this expression cassette is integrated into both chromosomes, e.g., both Z allosomes. In this instance, the genotype of the second type of engineered male chicken is Z²Z². The promoter driven toxin expression under specific transcription factor is activated by the expression from Z¹ and Z² expression cassettes, thereby generating a functional or active toxin that kills the cell that harbors both Z¹ and Z² expression cassettes.

In some instances, the Z¹ allosome is integrated with a transcription factor, and in this instance, the Z² allosome is integrated with the other expression cassette encoding an inducible promoter and a toxin located downstream of the inducible promoter. The promoter driven toxin expression under specific transcription factor is activated by the expression from Z¹ and Z², thereby generating a functional or active toxin that kills the cell that harbors both Z¹ and Z² expression cassettes.

In order to generate the second generation of chickens from the engineered female and male chickens, two different crosses may be employed. First, Z¹W female grandparent is crossed with Z²Z² male grandparent. This will generate viable Z²W female parent chicken because Z¹Z² male parent chicken expresses both expression cassettes and this generates functional or active toxin via an inducible promoter and a specific transcription factor system. Thus, male chicks are not viable. In some instances, the Z²W female parent chicken is crossed with Z¹Z¹ male chicken, thereby generating Z¹W female offspring chicken because Z¹Z² male offspring expresses both expression cassettes and this generates functional or active toxin via an inducible promoter and a specific transcription factor system. Thus, generation of single sex offspring, e.g., female chicken is achieved via promoter driven toxin under specific transcription factor. Crossing Z¹W with Z²Z² or Z²W with Z¹Z¹ will generate single sex offspring chicken, e.g., female chicken as long as Z¹ and Z² encode a transcription factor and an inducible promoter to express functional or active toxin.

In some instances, an engineered chicken is generated for generation of single sex offspring in the offspring generation. In this instance, the female parent chicken is engineered to harbor one or more expression cassette(s) encoding an inducible promoter and a toxin located downstream of the inducible promoter. This expression cassette is integrated into the female chicken chromosome. In some instances, the one or more expression cassette(s) is integrated into the Z allosome (called Z¹). In this instance, the genotype of female parent chicken is Z¹W. In this instance, the male parent chicken is engineered to harbor another expression cassette encoding a transcription factor that activate the inducible promoter to express the toxin, and, in this instance, this expression cassette is integrated into both chromosomes, e.g., both Z allosomes. In this instance, the genotype of the male parent chicken is Z²Z². The promoter driven toxin expression under specific transcription factor is activated by the expression from Z¹ and Z² expression cassettes, thereby generating a functional or active toxin that kills the cell that harbors both Z¹ and Z² expression cassettes. Thus, generation of single sex offspring, e.g., female chicken is achieved.

In other instances, an engineered chicken is generated for generation of single sex offspring in the offspring generation. In this instance, the female parent chicken is engineered to harbor an expression cassette encoding a transcription factor that activate an inducible promoter to express a toxin. This expression cassette is integrated into chicken chromosome. In some instances, this expression cassette is integrated into the Z allosome (called Z¹). In this instance, the genotype of the female parent chicken is Z¹W. In this instance, the male parent chicken is engineered to harbor another expression cassette encoding the inducible promoter and the toxin located downstream of the inducible promoter, and, in this instance, this expression cassette is integrated into both chromosomes, e.g., both Z allosomes. In this instance, the genotype of the male parent chicken is Z²Z². The promoter driven toxin expression under specific transcription factor is activated by the expression from Z¹ and Z² expression cassettes, thereby generating a functional or active toxin that kills the cell that harbors both Z¹ and Z² expression cassettes. Thus, generation of single sex offspring, e.g., female chicken is achieved.

### Mammals

Engineered mammals, e.g., cows, are generated for generation of single sex offspring, e.g., female cows. In some instances, a male cow in the first generation is engineered to harbor an expression cassette encoding an inducible promoter and a toxin located downstream of the inducible promoter. This expression cassette is integrated into the male cow chromosome. In some instances, this expression cassette is integrated into the Y allosome (called Y¹). In this instance, the genotype of the engineered male cow is XY¹.

The female cow in parental generation is engineered to harbor another expression cassette encoding a transcription factor that activates the inducible promoter to express the toxin, and, in some instances, this expression cassette is integrated into both chromosomes, e.g., both X allosomes. In this instance, the genotype of the engineered female cow is X²X². The promoter driven toxin expression under specific transcription factor is activated by the expression from Y¹ and X² expression cassettes, thereby generating a functional or active toxin that kills the cell that harbors both Y¹ and X² expression cassettes. Thus, generation of single sex offspring, e.g., female cow is achieved.

In some instances, the Y¹ allosome is integrated with another expression cassette encoding a transcription factor that activates an inducible promoter to express a toxin, and in this instance, the X² allosome is integrated with the other expression cassette encoding the inducible promoter and the toxin located downstream of the inducible promoter. The promoter driven toxin expression under specific transcription factor is activated by the expression from Y¹ and X² expression cassettes, thereby generating a functional or active toxin that kills the cell that harbors both Y¹ and X² expression cassettes.

In order to generate the offspring generation of cows from the engineered female and male cows, X²X² female parent is crossed with XY¹ male parent. This will generate viable XX² female offspring cow because X²Y¹ male offspring expresses both expression cassettes and this generates functional or active toxin via promoter driven toxin under specific transcription factor. Thus, male cows are not viable. Generation of single sex offspring, e.g., female cow, is achieved via an inducible promoter system with a specific transcription factor. This promoter driven toxin under specific transcription factor system for generation of single sex offspring can be applied to other animal, all of which are compatible with methods of the present disclosure and contemplated herein. Examples of animal include, but not limited to mammals, e.g., cow, mouse, rat, rabbit, guinea pig, bovine, chimpanzee, sheep, goat, and non-human primate.

### Methods of Generation of Single Sex Offspring Via Promoter Driven Toxin Under Specific Transcription Factor

By crossing the engineered animal as described in the present disclosure, generation of single sex offspring can be achieved. In one aspect, the present disclosure provides a method of producing a single sex population of non-human vertebrate animals, comprising the steps of: obtaining (i) a first non-human vertebrate comprising one or more nucleotide sequences integrated in one or more chromosomes comprises the following elements in 5′ to 3′ orientation: a first inactive promoter, operatively linked thereto a nucleotide sequence encoding for a toxin; obtaining (ii) a second non-human vertebrate animal comprising one or more nucleotide sequences integrated in one or more chromosomes comprising the following elements in 5′ to 3′ orientation: a second promoter, operatively linked thereto a transcription factor corresponding to the inactive promoter; and crossing the first non-human vertebrate animal and the second non-human vertebrate animals, wherein a resulting progeny, containing the first inactive promoter, the second promoter expressing the transcription factor corresponding to the first inactive promoter, and the corresponding transcription factor, is not viable; thereby creating a single sex population. In some embodiments, the chromosome is an autosome. In some embodiments, the chromosome is an allosome.

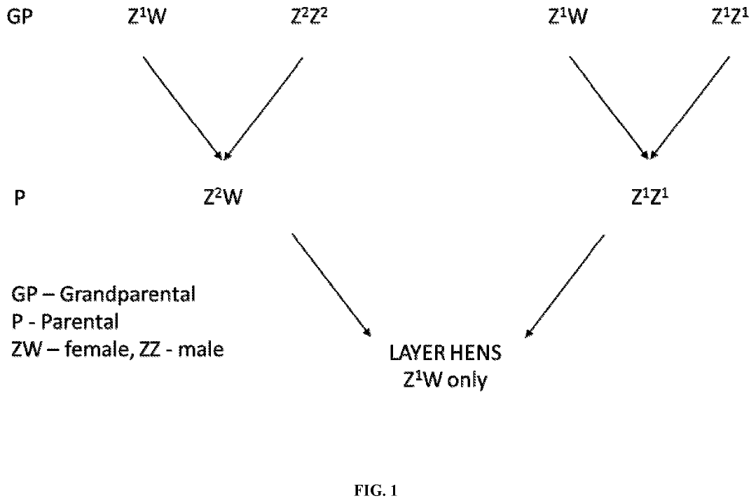

FIG. 1 is a genetic cross diagram showing how to generate single sex offspring in the third generation (Layer Hens) using protein splicing process involving split inteins system. In chicken, ZW indicates female while ZZ indicates male. As shown in FIG. 1, GP indicates grandparent generation (first generation or G1 or GP); P indicates parent generation (second generation or G2); and Layer Hens (third generation or G3). In this cross, Z¹ contains the half of a split intein fused to one half of a protein toxin. Z² contains the half of a split intein fused to the other half of the toxin. The cells that co-express the both halves of the intein-toxin fusion protein from chromosomes Z¹ and Z² contain functional toxin, which is a result of intein trans-splicing, and die.

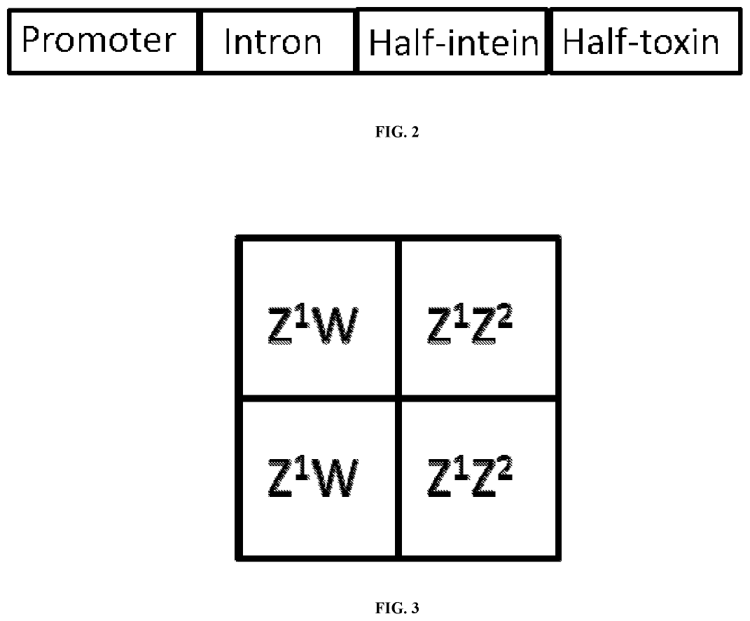

FIG. 2 shows an example of the DNA construct for generation of single sex offspring. In this instance, the DNA construct can be integrated into the Z¹ chromosome. The DNA construct integrated into the Z² chromosome is similarly constructed except the opposite half of the split intein and toxin integrated on the Z¹ chromosome are chosen. The DNA construct comprises a promoter. In some instances, the promoter is active during embryogenesis. In some instances, the DNA construct also contains an intron encoding sequence to facilitate the expression. Finally, the DNA construct contains half of a split intein (N-terminal split intein or C-terminal split intein) fused to one half of a protein toxin. The protein toxin includes, but not limited to, nucleases (e.g., Barnase, restriction endonuclease, CRISPR, etc.), ribosome toxins (e.g., diphtheria, ricin, abrin, pokeweed antiviral protein, etc.), protease (e.g., caspases, proteinase K, trypsin, chymotrypsin, papain, etc.), or other toxins capable of killing the host cell or endogenous protein whose overexpression is cytotoxic.

FIG. 3 depicts the Punnett Square of possible genotypic outcomes of 3rd generation from genetic crossing of second generation (Z²W and Z¹Z¹) chicken. The grandparental cross indicated by “GP” to generate the paternal female is arranged such that the desired offspring is a Z²W hen. The grandparental cross to generate the parental male is done such that the male harbors one promoter element of the type shown above on its Z¹ chromosome. When the parental chickens (Z²W and Z¹Z¹) as shown in FIG. 1 are crossed, there are four possible outcomes as shown in FIG. 3: Z¹W, Z¹W, Z¹Z², and Z¹Z².

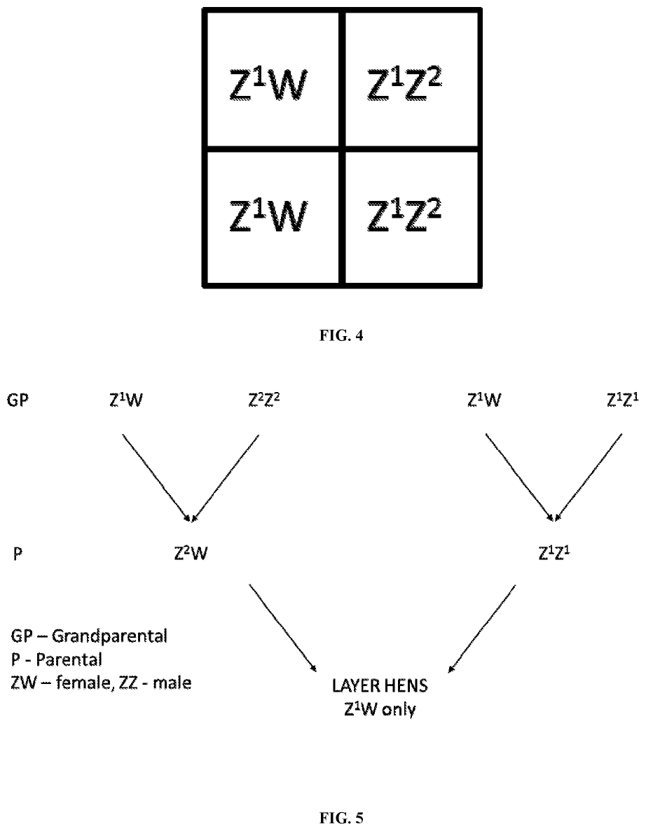

FIG. 4 shows the survival of G3 generation from FIG. 3. The Z¹Z² males, on the other hand, express both halves of the intein-toxin fusion protein. This fusion protein will undergo protein splicing, recreating a functional toxic protein which will kill the cells. Thus, the Z¹W females are viable.

FIG. 5 depicts a schematic of another genetic cross diagram to generate single sex offspring. Instead of using protein splicing process involving split-intein as in FIG. 1, this schematic uses a toxin gene driven by a promoter that is not normally active in chicken. In this approach, Z¹ contains a toxin encoding gene driven by a promoter that is inactive in chickens. Z² contains a transcription factor specifically designed to drive gene expression from the Z¹ promoter. In some instances, the transcription factor on Z² may be heterologous. In some instances, the promoter may also be conditionally activated by a small molecule. When Z¹ and Z² are in the same cell, the functional toxin is expressed and that cell dies.

While preferred embodiments of the present invention have been shown and described herein, such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

### EXAMPLES

The following examples are given for the purpose of illustrating various embodiments of the invention and are not meant to limit the present invention in any fashion. The present examples, along with the methods described herein are presently representative of preferred embodiments, are exemplary, and are not intended as limitations on the scope of the invention. Changes therein and other uses which are encompassed within the spirit of the invention as defined by the scope of the claims will occur to those skilled in the art.

### Example 1: Modified Allosomes Containing a Split Intein and Toxin DNA Construct

In this example, generation of single sex female layer hens is described. As shown in FIG. 1, GP indicates grandparent generation (first generation or G1 or GP); P indicates parent generation (second generation or G2); and Layer Hens (third generation or G3). In chicken, ZW indicates female while ZZ indicates male.

In the cross as shown in FIG. 1, Z¹ contains the half of a split intein fused to one half of a protein toxin. Z² contains the half of a split intein fused to the other half of the toxin. The cells that co-express the split intein from chromosomes Z¹ and Z² result in functional toxin and die.

The DNA construct integrated into the Z¹ chromosome is shown in FIG. 2. The DNA construct contains a promoter. In some instances, that promoter is active during embryogenesis. In some instances, the DNA construct also contains an intron encoding sequence to facilitate the expression. Finally, the DNA construct contains half of a split intein (n-intein or c-intein) fused to one half of a protein toxin. The protein toxin comprises Nucleases (e.g., Barnase, restriction endonuclease, CRISPR, etc.), Ribosome toxins (e.g., diphtheria, ricin, abrin, pokeweed antiviral protein, etc.), or protease (e.g., caspases, proteinase K, trypsin, chymotrypsin, papain, etc.). Other toxins capable of killing the host cell or endogenous protein whose overexpression is cytotoxic may be used.

The DNA construct integrated into the Z² chromosome is similarly constructed except the opposite half of the split intein and toxin integrated on the Z¹ chromosome are chosen.

The grandparental cross indicated by “GP” to generate the paternal female is arranged such that the desired offspring is a Z²W hen¹. The grandparental cross to generate the parental male is done such that the male harbors one promoter element of the type shown above on its Z¹ chromosome.

When the parental chickens (Z²W and Z¹Z¹) are crossed, there are four possible outcomes as shown in FIG. 3: Z¹W, Z¹W, Z¹Z², and Z¹Z².

The survival of G3 generation is shown in FIG. 4. The Z¹W females are viable. Female hens may express an intein-toxin fusion protein but absent its cognate pair so no splicing to reconstitute an active toxin occurs. This system has dual fail-safes since both intein components are required for splicing and both halves of the toxin must be present for its full reconstitution; thus, there is no chance of toxin generation in the Z¹W females. The Z¹Z² males, on the other hand, express both halves of the intein-toxin fusion protein. This fusion will splice in trans, recreating an active toxin which will kill the cells. An additional advantage of this system is that no protein will be present in the eggs laid by these layer hens since eggs for consumption are unfertilized.

In some instances, the promoter from which the split intein-toxin fusions are expressed is active during early embryogenesis—when expression is required for the system to work—and not thereafter when the expression is unnecessary.

### Example 2: Modified Allosomes Containing a Heterologous Promoter Toxin DNA Construct

In this example, the split intein system is replaced with a toxin gene driven by a promoter not normally active in chickens. The schematic diagram of this approach is shown in FIG. 5.

Z¹ indicate promoter driven toxin. In other words, Z¹ contains a toxin encoding gene driven by a promoter that is inactive in chickens. Z² contains a transcription factor specifically designed to drive gene expression from the Z¹ promoter. In some instances, the transcription factor on Z² may be heterologous. In some instances, the promoter may also be conditionally activated by a small molecule. When Z¹ and Z² are in the same cell, the toxin is expressed and that cell dies.

1.-88. (canceled)

89. A method of producing a single sex population of non-human vertebrate animals, the method comprising:
crossing (i) a first non-human vertebrate animal having a first genotype comprising a pair of heterozygous chromosomes comprising one or more nucleotide sequences encoding a first toxin fragment with (ii) a second non-human vertebrate animal having a second genotype comprising a pair of homozygous chromosomes comprising one or more nucleotide sequences encoding a second toxin fragment;
wherein a resulting progeny having a genotype comprising the first toxin fragment and the second toxin fragment is not viable;
thereby creating a single sex population.

90. The method of claim 89, wherein the chromosome is an autosome or an allosome.

91. (canceled)

92. The method of claim 89, wherein the non-human vertebrate animal is selected from the group consisting of cow, mouse, rat, rabbit, guinea pig, chicken, fish, bird, reptile, camelid, bovine, chimpanzee, sheep, goat, and non-human primate.

93. The method of claim 89, wherein the first toxin fragment and/or the second toxin fragment is a toxin selected from the group consisting of a nuclease, a ribosome toxin, and a protease.

94. The method of claim 93, wherein the nuclease comprises Barnase, an RNase, or a restriction endonuclease.

95. The method of claim 93, wherein the ribosome toxin comprises diphtheria, ricin, abrin, or pokeweed antiviral protein.

96. The method of claim 93, wherein the protease comprises a caspase, proteinase K, trypsin, chymotrypsin, or papain.

97. A method of producing a single sex population of non-human vertebrate animals, comprising the steps of:
(a) obtaining (i) a first non-human vertebrate animal, wherein one or more chromosomes comprise a first expression cassette comprising the following elements in 5′ to 3′ orientation: a first promoter, operatively linked thereto a nucleotide sequence encoding for a first toxin fragment and a first split intein fragment;
(b) obtaining (ii) a second non-human vertebrate animal, wherein one or more allosomes comprise a second expression cassette comprising the following elements in 5′ to 3′ orientation: a second promoter, operatively linked thereto a nucleotide sequence encoding for a second toxin fragment and a second split intein fragment;
(c) crossing the first non-human vertebrate animal and the second non-human vertebrate animals, wherein a resulting progeny containing the first expression cassette and the second expression cassette is not viable;
thereby creating a single sex population.

98. The method of claim 97, wherein the chromosome is an autosome or an allosome.

99. (canceled)

100. The method of claim 97, wherein the first and/or the second split intein fragment comprises an N-terminal and/or a C-terminal intein.

101.-107. (canceled)

108. The method of claim 97, wherein the first toxin fragment and/or the second toxin fragment is a toxin is selected from the group consisting of a nuclease, a ribosome toxin, and a protease.

109. The method of claim 108, wherein the nuclease comprises Barnase, an RNase, or a restriction endonuclease.

110. The method of claim 108, wherein the ribosome toxin comprises diphtheria, ricin, abrin, or pokeweed antiviral protein.

111. The method of claim 108, wherein the protease comprises a caspase, proteinase K, trypsin, chymotrypsin, or papain.

112.-118. (canceled)

119. A method of producing a single sex population of non-human vertebrate animals, comprising the steps of:
(a) obtaining (i) a first non-human vertebrate comprising one or more nucleotide sequences integrated in one or more chromosomes comprises the following elements in 5′ to 3′ orientation: a first inactive promoter, operatively linked thereto a nucleotide sequence encoding for a toxin;
(b) obtaining (ii) a second non-human vertebrate animal comprising one or more nucleotide sequences integrated in one or more chromosomes comprising the following elements in 5′ to 3′ orientation: a second promoter, operatively linked thereto a transcription factor corresponding to the inactive promoter; and
(c) crossing the first non-human vertebrate animal and the second non-human vertebrate animals, wherein a resulting progeny, containing the first inactive promoter, the second promoter expressing the transcription factor corresponding to the first inactive promoter, and the corresponding transcription factor, is not viable;
thereby creating a single sex population.

120. The method of claim 119, wherein the chromosome is an autosome or an allosome.

121. (canceled)

122. The method of claim 119, wherein the toxin is selected from the group consisting of a nuclease, a ribosome toxin, and a protease.

123. The method of claim 122, wherein the nuclease comprises Barnase, an RNase, or a restriction endonuclease.

124. The method of claim 122, wherein the ribosome toxin comprises diphtheria, ricin, abrin, or pokeweed antiviral protein.

125. The method of claim 122, wherein the protease comprises a caspase, proteinase K, trypsin, chymotrypsin, or papain.
